(19) United States
(12) Patent Application Publication
(10) Pub. No.: US 2004/0176430 A1
(43) Pub. Date: Sep. 9, 2004

(54) Propargyl-trifluoromethoxy-amino-benzothiazole derivatives

(76) Inventors: Jeffrey Sterling, Jerusalem (IL); Liat Hayardeny, Tel-Aviv (IL); Eliezer Falb, Givataim (IL); Yaacov Herzig, Raanana (IL); David Lerner, Jerusalem (IL)

Correspondence Address: John P. White, Cooper & Dunham LLP, 1185 Avenue of the Americas, New York, NY, 10036, US

(21) Appl. No.: 10/718,879

(22) Filed: Nov. 20, 2003

Related U.S. Application Data
(60) Provisional application No. 60/428,093, filed on Nov. 21, 2002.

(51) Int. Cl. C07D 277/82; A61K 31/428

(52) U.S. Cl. 514/367; 548/161

(57) ABSTRACT

The subject invention provides compounds having the structure:

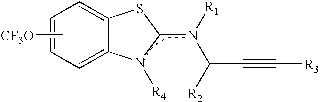

wherein R₁ is present or absent, and when present is H, C₁-C₆ alkyl, C₁-C₆ alkynyl, —(CH₂)_{y}S(CH₂)ₓCH₃, C₁-C₆ aminoalkyl, C₁-C₆ hydroxyalkyl or —(CH₂)ₙC(═O)(C₆H₄)(CH₂)R₂; R₂ is H or C₁-C₄ alkyl; R₃ is H or C₁-C₄ alkyl; R₄ is present or absent, and when present is H, C₁-C₆ alkyl, C₁-C₆ alkynyl, —(CH₂)_{y}S(CH₂)ₓCH₃, C₁-C₆ aminoalkyl, C₁-C₆ hydroxyalkyl or —(CH₂)ₙC(═O)(C₆H₄)(CH₂)R₂; wherein n is an integer from 1-6; wherein x is 0 or an integer from 1-5 and y is an integer from 1-5, such that x+y<6; at least one of R₁ or R₄ is present; the dashed line represents a bond between one of the nitrogen atoms and the intervening carbon atom; and any compound is charged when both R₁ and R₄ are present, or any specific enantiomer thereof or any pharmaceutically acceptable salt thereof, and a method for treating a neurologic disorder or multiple sclerosis by administering a therapeutically effective amount any of the compounds.

This application claims the benefit of U.S. Provisional Application No. 60/428,093, filed Nov. 21, 2002, the entire contents of which are hereby incorporated by reference.

Throughout this application various publications are referenced in parenthesis. The disclosures of these publications in their entireties are hereby incorporated by reference into this application in order to more fully describe the state of the art to which this invention pertains.

### BACKGROUND OF THE INVENTION

Neurologic disorders are becoming increasingly common in North America. For example, Parkinson's disease is the second most common neurologic disorder, affecting nearly 1 million people in North America. Thus, developing an effective treatment for neurologic disorders has become a high priority in the drug industry.

Neurologic disorders can generally be divided into two groups based on their physiological and pathological characteristics. Parkinson's disease, Alzheimer's disease, Huntington's disease and amyotrophic lateral sclerosis (ALS or Lou Gehrig's disease) are all progressive disorders (i.e., their symptoms are not apparent until months or more commonly years after the disease has begun), caused by an initial reduction of neuronal function, followed by a complete loss of function upon neuronal death. In addition, these progressive neurologic disorders are characterized by the presence of protein aggregates that are believed to hamper cellular functions (e.g., neurotransmission), and may ultimately result in cell death (Sasaki et al., Am. J. Pathol., 153:1149-1155 [1998]).

Multiple sclerosis is a disorder of the central nervous system, which is slowly progressive and is characterized by disseminated patches of demyelination in the brain and spinal cord, resulting in multiple and varied neurologic symptoms and signs, usually with remissions and exacerbations. The cause is unknown but an immunologic abnormality is suspected (THE MERCK MANUAL, 17th EDITION, 1999 MERCK & CO.). Several different drug therapies are currently being investigated.

While the aforementioned disorders are all slowly progressive, neurological dysfunction can also be caused by a more abrupt event such as an infarction of brain tissue, or stroke. Brain stroke is the third leading cause of death in the developed countries. Survivors often suffer from neurological and motor disabilities. The majority of central nervous system (“CNS”) strokes are regarded as localized tissue anemia following obstruction of arterial blood flow which causes oxygen and glucose deprivation. R(+)-N-propargyl-1-aminoindan has been shown to be an effective treatment for stroke and traumatic brain injury (U.S. Pat. No. 5,744,500).

A series of propargylamines, including Selegiline and Rasagiline, have been shown to prevent apoptosis in dopamine neurons in Parkinson's models (Naoi, M. et al. J. Neural Transmission (2002) 109: 607-721). N-Propargyl-1-Aminoindan has recently been suggested as being useful for treating Parkinson's disease, dementia and depression (U.S. Pat. No. 5,453,446). The neuroprotective activity of these molecules is attributed by some to the presence of the propargyl moiety. The mechanism by which the propargyl moiety may confer neuroprotection is not fully understood. However, it is clear that the mechanism involves a complex set of neurochemical events including alterations in Bcl-2, GAPDH, SOD and catalase (Youdim, M. B. H. Cell. Mol. Neurobiol. (2001) 21(6): 555-573).

Riluzole (6-trifluoromethoxy-2-amino-benzothiazole) has recently emerged as a pharmacological agent potentially useful to slow down the evolution of amyotrophic lateral sclerosis, a neurologic disorder. (Ben Simon et al., New Engl. J. Med., 330:585-91 (1994)). PCT International Publication No. WO 01/95907 suggests the use of Riluzole for treatment or prevention of the onset of symptoms of multiple sclerosis. PCT International Publication No. WO 00/74676 suggests the use of Riluzole for treatment of multiple sclerosis either alone or in combination with other drugs.

6-trifluoromethoxy-2-amino-benzothiazole, PK 26124, RP 54274, Riluzole, was synthesized for the first time by a Russian group (J. Gen. Chem. USSR, 1963, 33, 2240-2246, U.S. Pat. No. 2,822,359; Ch.A., 52, 8570d 1958) as a part of their investigation on the influence of electronegative substituents at the 6 position of the benzothiazole nucleus on the color of diazastyryl bases.

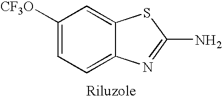

2-Aminobenzothiazoles exist in tautomeric forms (where the proton is shifted between the 2-amino group and the ring nitrogen). When this process is blocked by complete alkylation of the 2-amino group or by alkylation of the ring nitrogen to give for instance 2-imino-3-methylbenzothiazoline, the depressant effect of the 2-aminobenzothiazole is changed to stimulation of the CNS. (Domino, E. F. et al., J. Pharmacol. Exp. Ther., (1952) 105: 486-497) Domino and co-workers have also shown that the azole structure is essential for the paralyzing effect of the benzazoles, since opening that ring system resulted in convulsing activity. None of the benzazoles had any curare-like action in doses producing paralysis.

A group of 6-trifluoromethoxy-2-amino-benzothiazoles bearing a variety of substituents on the amino group was generically disclosed in European Patent No. EP 282 971 and U.S. Pat. Nos. 4,826,860, 4,918,090, and 4,971,983, as effective for treating cerebrovascular disorders.

U.S. Pat. No. 4,535,088 discloses propynylaminothiazole derivatives having anti-fungal and/or anti-microbial activity.

The present invention provides novel derivatives of propargyl-trifluoromethoxy-amino-benzothiazole which are effective at treating neurologic disorders, including Parkinson's disease, and multiple sclerosis.

### SUMMARY OF THE INVENTION

The subject invention provides compounds having the structure:

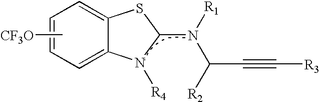

wherein

R₁ is present or absent, and when present is H, C₁-C₆ alkyl, C₁-C₆ alkynyl, —(CH₂)_{y}S(CH₂)ₓCH₃, C₁-C₆ aminoalkyl, C₁-C₆ hydroxyalkyl or —(CH₂)ₙC(═O)(C₆H₄)(CH₂)R₂;

R₂ is H or C₁-C₄ alkyl;

R₃ is H or C₁-C₄ alkyl;

R₄ is present or absent, and when present is H, C₁-C₆ alkyl, C₁-C₆ alkynyl, —(CH₂)_{y}S(CH₂)ₓCH₃, C₁-C₆ aminoalkyl, C₁-C₆ hydroxyalkyl or —(CH₂)ₙC(═O)(C₆H₄)(CH₂)R₂;

wherein n is an integer from 1-6;

wherein x is 0 or an integer from 1-5 and

y is an integer from 1-5, such that x+y<6;

at least one of R₁ or R₄ is present;

the dashed line represents a bond between one of the nitrogen atoms and the intervening carbon atom; and

any compound is charged when both R₁ and R₄ are present,

or any specific enantiomer thereof or any pharmaceutically acceptable salt thereof.

The subject invention also provides a method for treating a subject afflicted with a neurologic disorder comprising administering to the subject a therapeutically effective amount of any of the compounds of the invention or pharmaceutically acceptable salts thereof so as to thereby treat the neurologic disorder in the subject.

The subject invention also provides a method for treating a subject afflicted with multiple sclerosis comprising administering to the subject a therapeutically effective amount of any of the compounds of the invention or pharmaceutically acceptable salts thereof so as to thereby treat multiple sclerosis in the subject.

### DETAILED DESCRIPTION OF THE FIGURES

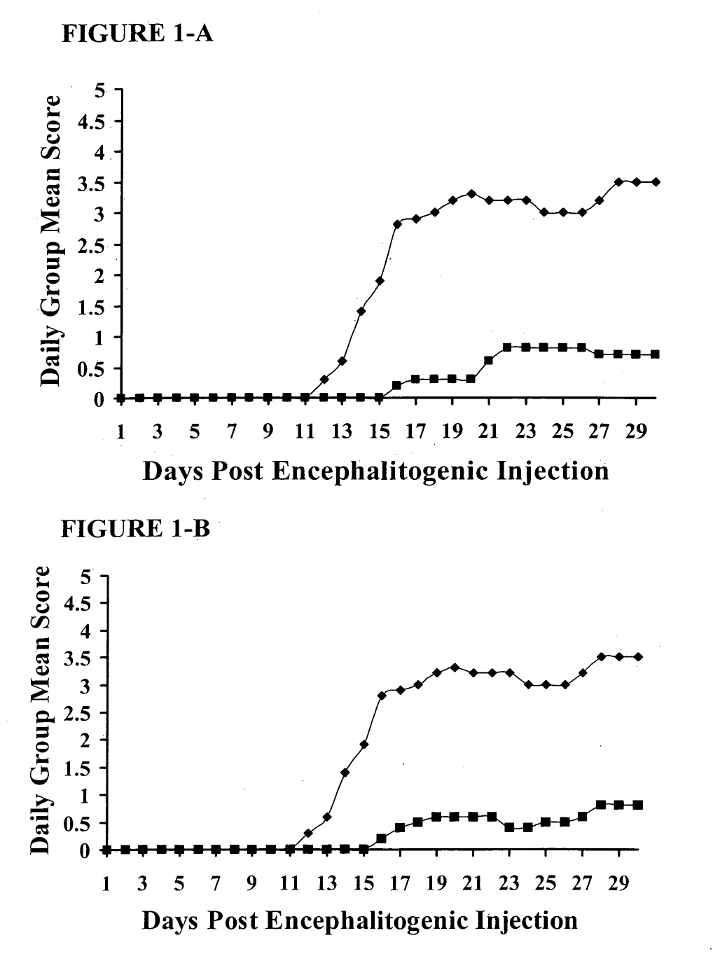

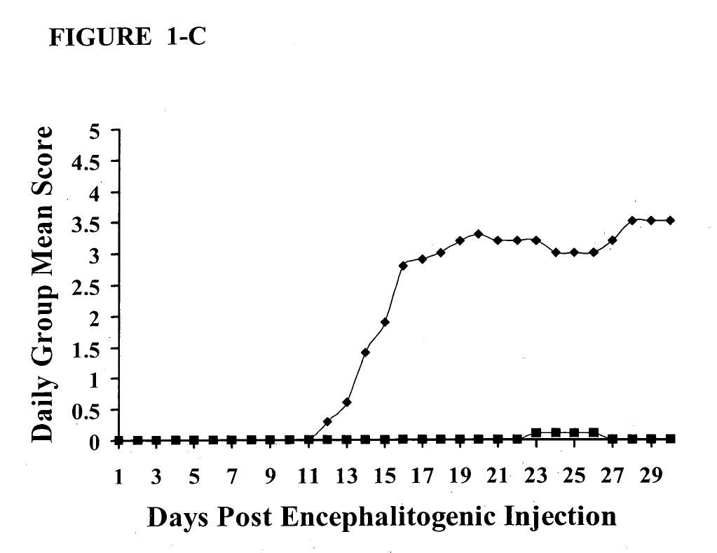

FIG. 1-A shows the daily EAE GMS for compound 3 (10 mg/kg twice daily).

—♦— indicates the control group (PBS);

—▪— indicates the group under study.

FIG. 1-B shows the daily EAE GMS for Glatiramer Acetate (75 μg/mouse).

—♦— indicates the control group (PBS);

—▪— indicates the group under study.

FIG. 1-C shows the daily EAE GMS for Glatiramer Acetate (75 μg/mouse)+Compound 3 (10 mg/kg twice a day).

—♦— indicates the control group (PBS);

—▪— indicates the group under study.

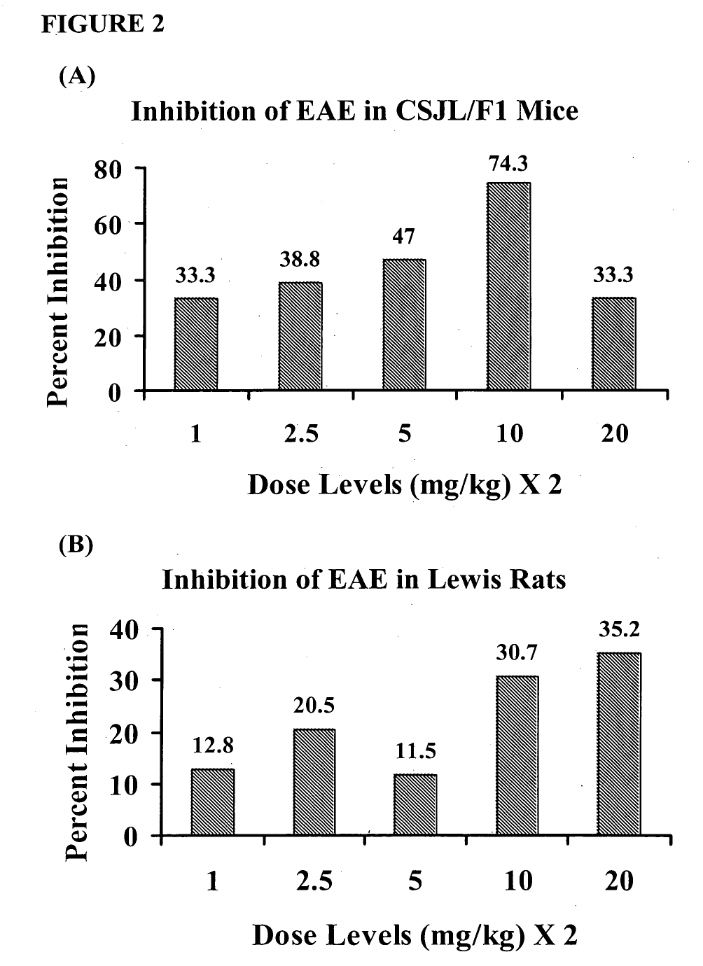

FIG. 2 shows dose response of EAE MMS (averages from several experiments) for Compound 3

(a) in CSJL/F1 mice

(b) in Lewis rats

### DETAILED DESCRIPTION OF THE INVENTION

The subject invention provides compounds having the structure (Formula I):

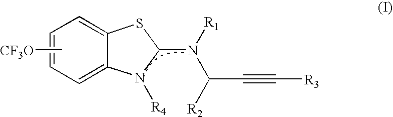

wherein

R₁ is present or absent, and when present is H, C₁-C₆ alkyl, C₁-C₆ alkynyl, —(CH₂)_{y}S(CH₂)ₓCH₃, C₁-C₆ aminoalkyl, C₁-C₆ hydroxyalkyl or —(CH₂)ₙC(═O)(C₆H₄)(CH₂)R₂;

R₂ is H or C₁-C₄ alkyl;

R₃ is H or C₁-C₄ alkyl;

R₄ is present or absent, and when present is H, C₁-C₆ alkyl, C₁-C₆ alkynyl, —(CH₂)_{y}S(CH₂)ₓCH₃, C₁-C₆ aminoalkyl, C₁-C₆ hydroxyalkyl or —(CH₂)ₙC(═O)(C₆H₄)(CH₂)R₂;

wherein n is an integer from 1-6;

wherein x is 0 or an integer from 1-5 and

y is an integer from 1-5, such that x+y<6;

at least one of R₁ or R₄ is present;

the dashed line represents a bond between one of the nitrogen atoms and the intervening carbon atom; and

any compound is charged when both R₁ and R₄ are present,

or any specific enantiomer thereof or any pharmaceutically acceptable salt thereof.

In a first embodiment of the above compounds, at least one of R₁ or R₄ is —(CH₂)ₙC(═O)(C₆H₄)(CH₂)R₂.

In a second embodiment of the above compounds, at least one of R₁ and R₄ is —(CH₂)_{y}S(CH₂)ₓCH₃.

In a third embodiment of the above compounds, the subject invention provides compounds having the structure (Formula II):

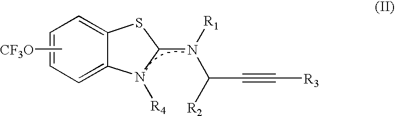

wherein

R₁ is present or absent, and when present is H or C₁-C₄ alkyl;

R₂ is H or C₁-C₄ alkyl;

R₃ is H or C₁-C₄ alkyl;

R₄ is present or absent, and when present is H or C₁-C₄ alkyl;

at least one of R₁ or R₄ is present;

the dashed line represents a bond between one of the nitrogen atoms and the intervening carbon atom; and

any compound is charged when both R₁ and R₄ are present,

or any specific enantiomer thereof or any pharmaceutically acceptable salt thereof.

In what follows, the phrase “compounds which are represented by Formula II” refers to compounds which are encompassed by the preceding description.

In a second embodiment of the compounds represented by Formula II, the compounds have the structure:

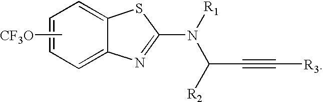

In a third embodiment of the compounds have the structure:

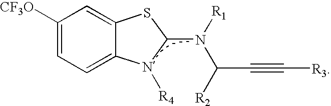

In a fourth embodiment, the compounds have the structure:

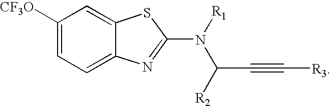

In a further embodiment of any of the aforementioned compounds represented by Formula II, at least one of R₁, R₂ and R₃ is C₁-C₄ alkyl.

In another embodiment of any of the aforementioned compounds represented by Formula II, R₁ is H or methyl; R₂ is H or methyl; and R₃ is H or methyl, or a pharmaceutically acceptable salt thereof.

In another embodiment of the third embodiment of the compounds represented by Formula I or the third embodiment of the compounds represented by Formula II, R₁ is absent and R₄ is present.

In a further aspect of the above embodiment, R₁ is absent and R₄ is methyl.

In another embodiment of any of the aforementioned compounds represented by Formula II, the chiral carbon is in the R configuration.

In another embodiment of any of the aforementioned compounds represented by Formula II, the chiral carbon is in the S configuration.

In a further embodiment the subject invention provides the pharmaceutically acceptable salt of any of the aforementioned compounds, wherein the salt is the chloride, mesylate, maleate, fumarate, tartarate, hydrochloride, hydrobromide, esylate, p-toluenesulfonate, benzoate, acetate, phosphate or sulfate salt.

In one embodiment of the first embodiment of the compounds represented by Formula I, the compound has the structure:

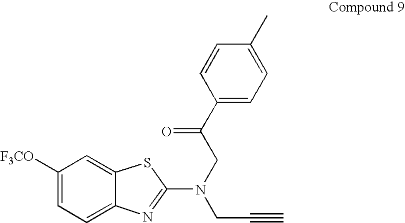

In one embodiment of the compounds represented by Formula I, the compound has the structure:

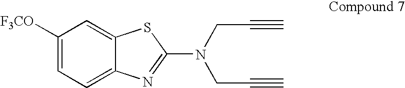

In one embodiment of the second embodiment of the compounds represented by Formula I, the compound has the structure:

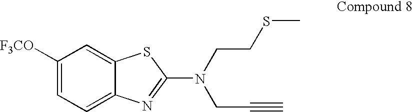

In another embodiment of the second embodiment of the compounds represented by Formula I, the compound has the structure:

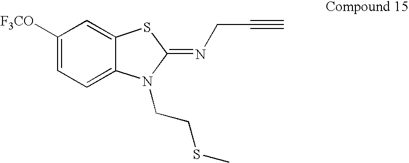

In one embodiment of the compounds represented by Formula I, the compound has the structure:

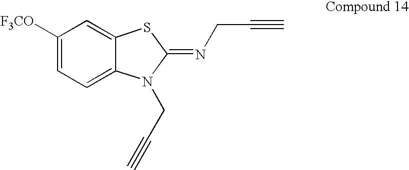

In another embodiment of the first embodiment of the compounds represented by Formula I, the compound has the structure:

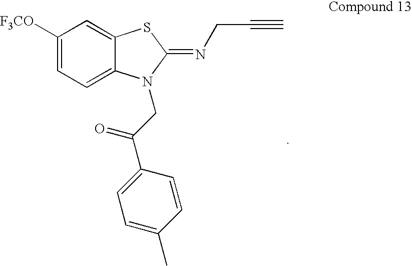

In another embodiment of the compounds represented by the fourth embodiment of compounds represented by Formula II, the compound has the structure:

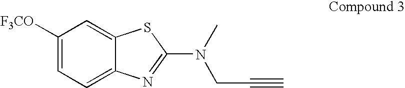

In a further embodiment, the subject invention provides the hydrochloride salt of the above compound.

In another embodiment of the compounds represented by the fourth embodiment of compounds represented by Formula II, the compound has the structure:

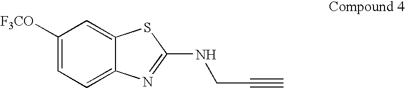

In a further embodiment, the subject invention provides the hydrochloride salt of the above compound.

In another embodiment of the compounds represented by the fourth embodiment of compounds represented by Formula II, the compound has the structure:

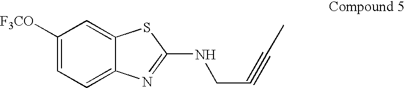

In a further embodiment, the subject invention provides the hydrochloride salt of the above compound.

In another embodiment of the compounds represented by the fourth embodiment of compounds represented by Formula II, the compound has the structure:

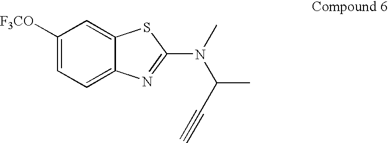

In a further embodiment, the subject invention provides the hydrochloride salt of the above compound.

In another embodiment of the compounds represented by the second embodiment of compounds represented by Formula II, the compound has the structure:

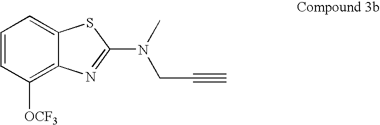

In a further embodiment, the subject invention provides the hydrochloride salt of the above compound.

In another embodiment of the compounds represented by the third embodiment of compounds represented by Formula II, the compound has the structure:

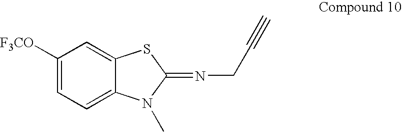

In a further embodiment, the subject invention provides the hydrochloride salt of the above compound.

In another embodiment of the compounds represented by Formula II, the compound has the structure:

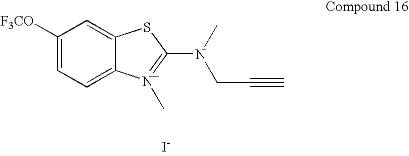

In another embodiment of the compounds represented by Formula II, the compound has the structure:

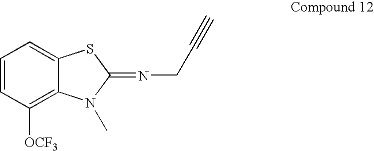

The subject invention also provides a method for treating a subject afflicted with a neurologic disorder comprising administering to the subject a therapeutically effective amount of any of the aforementioned compounds represented by Formula I or a pharmaceutically acceptable salt thereof, so as to thereby treat the neurologic disorder in the subject.

In one embodiment of the above method, the disorder is Parkinson's Disease, Alzheimer's Disease, amyotrophic lateral sclerosis, stroke, a neuromuscular disorder, schizophrenia, cerebral infarction, head trauma, glaucoma, facialis or Huntington's Disease.

In a further embodiment of the above method, the therapeutically effective amount is from about 1 to about 1000 mg/day.

The subject invention also provides a method for treating a subject afflicted with multiple sclerosis comprising administering to the subject a therapeutically effective amount of any of the aforementioned compounds represented by Formula I or a pharmaceutically acceptable salt thereof so as to thereby treat multiple sclerosis in the subject.

In one embodiment, the above method further comprises administering to the subject a therapeutically effective amount of levodopa, glatiramer acetate, interferon beta-1b, interferon beta-1a, steroids or Mitoxantrone (Novantrone).

In another embodiment of the above method, the therapeutically effective amount is from about 1 to about 1000 mg/day.

In one embodiment of either of the above methods the therapeutically effective amount of the compound is administered by injection, systemically, orally or nasally.

The subject invention also provides the use of any of the aforementioned compounds for manufacturing a medicament useful for treating a neurologic disorder in a subject.

In one embodiment of the above use, the neurologic disorder is Parkinson's Disease, Alzheimer's Disease, amyotrophic lateral sclerosis, stroke, a neuromuscular disorder, schizophrenia, cerebral infarction, head trauma, glaucoma, facialis or Huntington's Disease.

The subject invention also provides the use of any of the aforementioned compounds for manufacturing a medicament useful for treating multiple sclerosis in a subject.

In one embodiment of the above use, the medicament further comprises levodopa, glatiramer acetate, interferon beta-1b, interferon beta-1a, steroids or Mitoxantrone (Novantrone).

As noted above, the compounds of this invention may be used therapeutically in addition to currently available treatments for neurologic disorders. For instance, the compounds of the invention may be used in addition to levodopa therapy for Parkinson's disease or in addition to glatiramer acetate (the drug substance of Copaxone), interferon beta1b, interferon beta-1a, steroids or Mitoxantrone (Novantrone).

The subject invention also provides the use of any of the aforementioned compounds for manufacturing a medicament in a package having instructions for administration of the medicament to treat a neurologic disorder in a subject.

The subject invention also provides a method for destroying or inhibiting the proliferation of microbes or fungus which comprises contacting the microbes or fungus with a composition comprising any of the aforementioned compounds and an acceptable carrier.

The subject invention also provides a pharmaceutical composition comprising any of the aforementioned compounds and a pharmaceutically acceptable carrier.

In one embodiment the pharmaceutical composition further comprises a therapeutically effective amount of levodopa, glatiramer acetate, interferon beta-1b, interferon beta-1a, steroids or Mitoxantrone (Novantrone).

In one embodiment the pharmaceutical composition further comprises a therapeutically effective amount of glatiramer acetate.

The subject invention also provides a process for the manufacture of the above pharmaceutical composition comprising admixing any of the aforementioned compounds with a pharmaceutically acceptable carrier.

The subject invention also provides a packaged pharmaceutical composition for treating a neurologic disorder in a subject comprising: (a) any of the aforementioned pharmaceutical compositions; and (b) instructions for using the composition for treating the neurologic disorder in the subject.

The subject invention also provides a process of manufacturing compound of Structure II, comprising the steps of:

(a) reacting

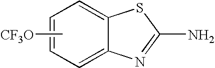

under suitable conditions with an amine exchanging agent in the presence of solvent to provide:

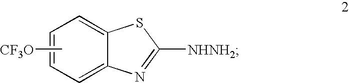

(b) treating 2 with a chlorinating agent to provide

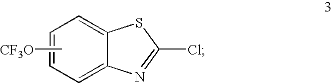

(c) reacting 3 with

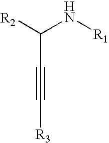

to provide

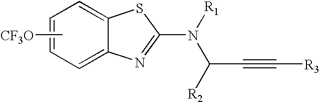

wherein

R₁ is present or absent, and when present is H or C₁-C₄ alkyl;

R₂ is H or C₁-C₄ alkyl;

R₃ is H or C₁-C₄ alkyl; and

(d) optionally alkylating the product of step (c), wherein R₁ is H, to provide the compound.

In one embodiment, the above process further comprises reacting the product of step (c), wherein R₁, R₂ and R₃ are each H, with 2-bromo-4′-methylacetophenone in a polar solvent in the presence of a base to produce a compound having the structure:

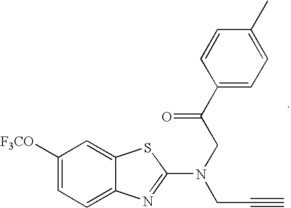

In one embodiment the polar solvent is acetonitrile and the base is potassium carbonate.

In a further embodiment of the above process for manufacturing compounds of structure II, the process further comprises reacting the product of step (c), wherein R₁, R₂ and R₃ are each H, with propargyl bromide in a polar solvent in the presence of a base to produce a compound having the structure:

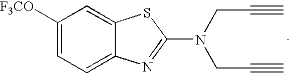

In one embodiment, the polar solvent is acetonitrile and the base is potassium carbonate.

In a further embodiment of the above process for manufacturing compounds of structure II, the process further comprises reacting the product of step (c), wherein R₁, R₂ and R₃ are each H, with 2-chloroethyl methylsulfide in a polar solvent in the presence of a base, to produce a compound having the structure:

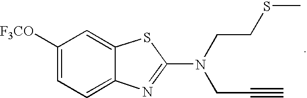

In one embodiment, the polar solvent is acetonitrile and the base is potassium carbonate.

In one embodiment of the above process for manufacturing compounds of structure II, the amine exchanging agent is a mixture of aqueous NH₂NH₂ and hydrazinium sulfate in ethylene glycol.

In another embodiment of the above process, the chlorinating agent is SOCl₂.

In another embodiment of the above process, R₁ is C₁-C₄ alkyl and R₂ and R₃ are H.

In a further embodiment of the above process for manufacturing compounds of structure II, the alkylating agent in step (d) is methyliodide or dimethyl sulfate.

The subject invention also provides a process of manufacturing a compound having the structure:

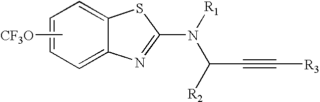

wherein

R₁ is C₁-C₄ alkyl;

R₂ is H or C₁-C₄ alkyl; and

R₃ is H or C₁-C₄ alkyl,

comprising reacting a compound having the structure:

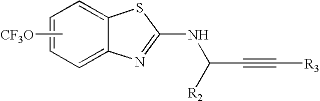

with R₁X in a polar solvent in the presence of a base, wherein X is a halogen atom, to produce the compound.

In one embodiment of the above process, the polar solvent is acetonitrile and the base is potassium carbonate.

The subject invention also provides a process of manufacturing a compound having the structure:

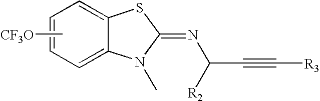

wherein

R₂ is H or C₁-C₄ alkyl; and

R₃ is H or C₁-C₄ alkyl,

comprising,

a) reacting

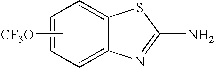

under suitable conditions with a methylating agent, in the presence or absence of solvent to provide:

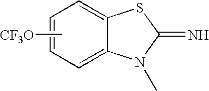

b) reacting the product of step a) with

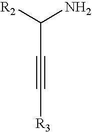

in the presence of p-toluenesulfonic acid to provide the compound.

In one embodiment of the above process, the product of step (b) is further alkylated with an alkylating agent to provide a compound having the structure:

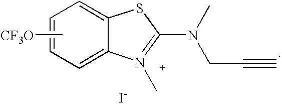

In an additional embodiment of the above process, the methylating agent in step (a) is methyliodide or dimethyl sulfate.

In a further embodiment of the above process, the methylating agent is methyliodide.

The subject invention further provides a process of manufacturing compound 14 comprising reacting a compound having the structure:

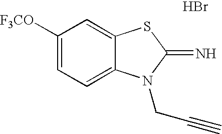

with propargylamine and p-TsOH in toluene to produce the compound.

The subject invention further provides a process of manufacturing compound 15 comprising reacting a compound having the structure:

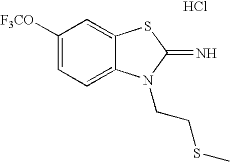

with propargylamine and p-TsOH in toluene to produce the compound.

The subject invention further provides a process of manufacturing compound 9 comprising reacting a compound having the structure:

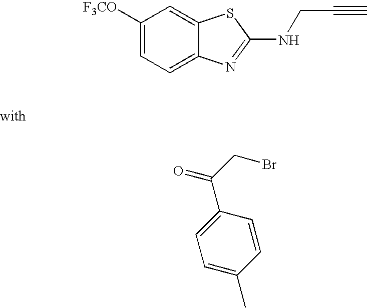

in a polar solvent to produce the compound.

In one embodiment of the above process, the polar solvent is acetonitrile.

In the above embodiments, when both R₁ and R₄ are alkyl the compound is positively charged and is present as a quaternary ammonium salt.

Those skilled in the art will readily expect all of the disclosed compounds to have biological activity similar to the tested compounds. In particular, compounds with both small groups, such as H and methyl, and bulky groups, such as p-tolyl-ethanone, in the R₄ position have biological activity in the described results. Consequently, compounds with the described intermediate sized groups at the R₄ position are reasonably expected to have biological activity. Compounds with small groups, such as methyl, in the R₁ position have biological activity. Compounds with bulky groups, such as p-tolyl-ethanone, in the R₁ position have biological activity. Consequently, compounds with the described intermediate sized groups at the R₁ position are reasonably expected to have biological activity. Compounds with small groups, such as H, in the R₂ or R₃ positions have biological activity. Consequently, compounds with the described C₁-C₄ alkyl at the R₂ or R₃ position are reasonably expected to have biological activity.

Those skilled in the art will be familiar with the fact that some compounds of the formula (I) can exist as tautomers. The compounds of the formula (I) are therefore also to be understood as meaning, hereinabove, and hereinbelow, the relevant tautomers, even when the latter are not mentioned specifically in each individual case. This invention also relates to the use of all such tautomers and mixtures thereof.

The subject invention further provides any of the aforementioned compounds for use as antimicrobial and/or antifungal agents.

The invention further contemplates the use of prodrugs which are converted in vivo to the therapeutic compounds of the invention (see, e.g., R. B. Silverman, 1992, “The Organic Chemistry of Drug Design and Drug Action”, Academic Press, Chapter 8, the entire contents of which are hereby incorporated by reference). Such prodrugs can be used to alter the biodistribution (e.g., to allow compounds which would not typically enter the reactive site of the protease) or the pharmacokinetics of the therapeutic compound.

As set out above, certain embodiments of the present compounds can contain a basic functional group, such as amino or alkylamino, and are thus capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The term “pharmaceutically acceptable salts” as used herein also includes a quaternary ammonium salt.

It will be noted that the structure of some of the compounds of this invention includes asymmetric carbon atoms and thus occur as racemates and racemic mixtures, single enantiomers, diastereomeric mixtures and individual diastereomers. All such isomeric forms of these compounds are expressly included in this invention. Each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis.

When the compounds of the present invention are administered as pharmaceuticals, to humans and mammals, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a compound(s) of the present invention within or to the subject such that it can performs its intended function. Typically, such compounds are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, pills, tablets, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert dilutents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert dilutents, the oral compositions can also include adjuvants such as wetting agents, emulsifying and suspending agents, sweetening, flavoring, coloring, perfuming and preservative agents.

Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The phrases “parenteral administration” and “administered parenterally” as used herein means modes of administration, other than enteral and topical administration, usually by injection, and includes, without limitation, intravenous, intramuscular, intraarterial, intrathecal, intracapsular, intraorbital, intracardiac, intradermal,intraperitoneal, transtracheal, subcutaneous, subcuticular, intraarticular, subcapsular, subarachnoid, intraspinal and intrasternal injection and infusion.

The phrases “systemic administration,” “administered systematically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above.

If desired, the effective daily dose of the active compound may be administered as two, three, four, five, six or more sub-doses administered separately at appropriate intervals throughout the day, optionally, in unit dosage forms.

It is noted that the compounds of the invention can prevent neuronal death and improve the outcome in various models resembling human degenerative disorders.

The phrase “neurologic disorder” as used herein refers to a disorder whose adverse affects are localized in the nervous system.

The term “neurotrauma” as used herein, refers to damage to the central or peripheral nervous system caused by a traumatic event, such as head trauma, spinal trauma, neurotoxic injury, stroke, ischemia, hypoxia, or anoxia.

The term “stroke” or “ischemic stroke” as used herein means a brain infarct manifested by neurologic deficits. “Stroke” may refer to a “stroke in evolution” in which the infarction is still enlarging or a “completed stroke” in which the infarction size is no longer growing. (THE MERCK MANUAL, 17th EDITION, 1999 MERCK & CO.)

The phrase “treatment of stroke” as used herein is meant to include the treatment of the brain infarction per se or the treatment of the symptoms caused by the brain infarction. Such symptoms may include neurological deficits, cognitive disturbances, brain edema, decreased cerebral blood flow, catecholamine fluctuations, or neurological or motor disabilities.

The invention is further illustrated by the following examples which in no way should be construed as being further limiting. The contents of all references, pending patent applications and published patent applications, cited throughout this application, including those referenced in the background section, are hereby incorporated by reference. It should be understood that the models used throughout the examples are accepted models and that the demonstration of efficacy in these models is predictive of efficacy in humans.

This invention will be better understood from the Experimental Details which follow. However, one skilled in the art will readily appreciate that the specific methods and results discussed are merely illustrative of the invention as described more fully in the claims which follow thereafter.

### Experimental Details

A synthesis scheme for the preparation of a group of compounds of the invention is outlined below in Scheme I.

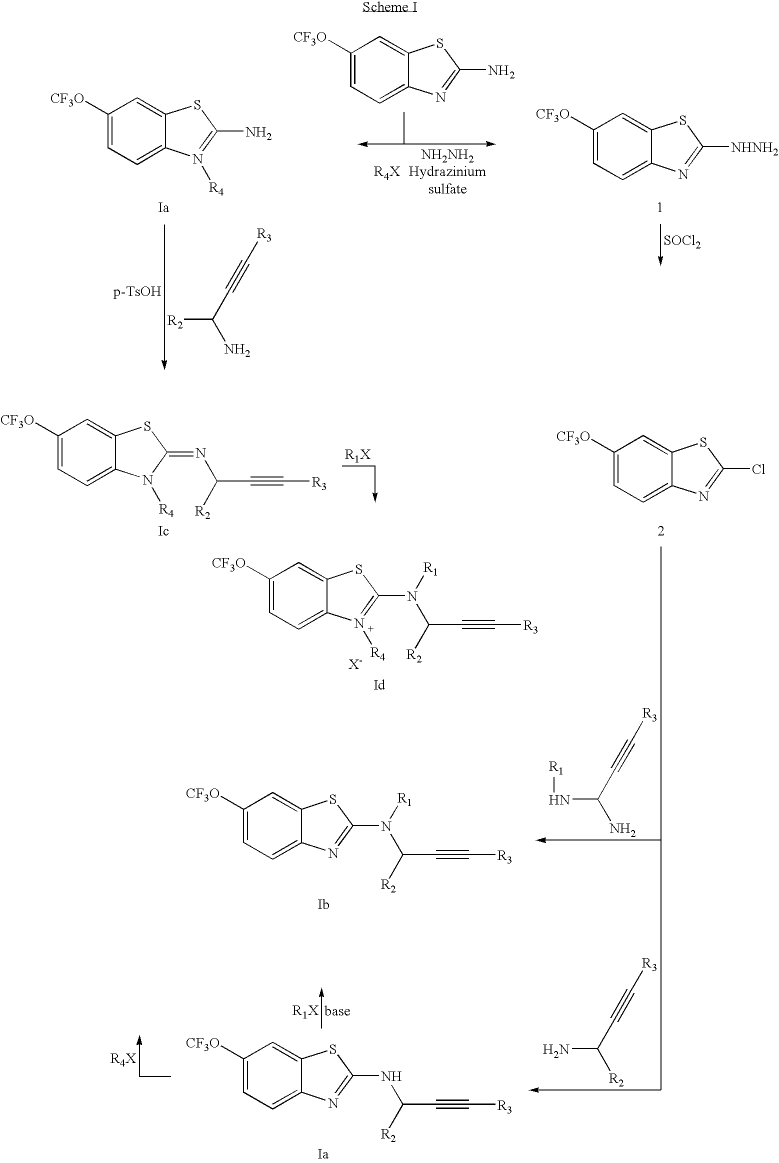

Benzothiazoles Ia and Ib were prepared by converting 6-trifluoromethoxy 2-amino benzothiazole to the corresponding 2-hydrazino analogue by direct exchange amination (C. J. Barnett and J. C. Smirz, Organic. Prep. Proc. Int. 6(4), 1974, 179-182) using a mixture of aqueous hydrazine and hydrazinium sulfate in ethylene glycol, followed by substituting the 2-hydrazino group with a chlorine atom by reacting 1 with SOCl₂, to give 2 (Barry A. Dreikorn and Paul Unger, J. Heterocyclic Chem., 26, 1989, 1735-1737), and finally reacting the latter with substituted propargylamines, to afford the compounds of formula Ia and Ib. Compounds Ib were also prepared by reacting Ia with alkylating agents R₁X in a polar solvent, e.g. acetonitrile, in the presence of a base, e.g. potassium carbonate, at ambient temperature. 2-Imino derivatives Ic were prepared by regioselective alkylation of 6-trifluoromethoxy-2-amino-benzothiazole with alkylating agents R₄X to give 3-alkyl-2-imino-6-trifluoromethoxy-benzothiazoline 1a (Patrick Jimonet et al., J. Med. Chem. 1999, 42, 2828-2843) in high yield, followed by propargylation of the 2-imino moiety by reacting la with propagylamines in the presence of p-TsOH (CH patent 667091 A5) either in toluene as the reaction solvent or neat with excess amine. Compounds Ic were also prepared as their acid addition salts by alkylation of Ia by using an excess of an alkylating agent R₄X, in a suitable solvent, e.g. methyl ethyl ketone (A. R. Katritzky et al. J. Chem. Soc. Perkin Trans. I, 1987, 2539-2541) or acetonitrile and concentrating the reaction mixture, treating the residue with toluene and filtering the salt. Alkylating compounds Ic (as their free base) with an excess of alkylating agents R₁X provided the quaternary 3-alkyl-benzothiazolium salt Id, isolated by filtration.

### EXAMPLE 1

### (6-Trifluoromethoxy-benzothiazol-2-yl)-hydrazine 1

A suspension of 6-trifluoromethoxy-2-amino-benzothiazole (crude, 1 g, 4.27 mmol), hydrazinium sulfate (NH₂NH₂.H₂SO₄, 0.85 g, 6.53 mmol) and hydrazine hydrate (˜82% aqueous solution, 2.7 ml, ˜45 mmol) in ethylene glycol (10 ml) was heated with stirring under N₂ atm. at 140° C. for 2.5 h. When the solution was cooled to rt a white solid precipitated, water (10 ml) was added to complete precipitation, the product filtered, washed with water and vacuum dried to give 1 (0.7 g, 65%) as a white powder. ¹H-NMR (CD₃CN) δ 7.70 (br s, 1H, NH), 7.63 (dq, 1H, J=2, 1 Hz, H-7), 7.41 (d, 1H, J=9 Hz, H-4), 7.18 (ddq, 1H, J=9, 2, 1 Hz, H-5), 4.50 (br s, 2 H, NH₂); ¹³C (CD₃CN) δ 115.81, 120.35, 120.58 (3×CH, C-4, C-5, C-7), 120.93 (CF₃O), 133.01, 143.89, 153.47 (3×C, C-3a, C-7a, C-6), 176.27 (C-2); MS (CI)(NH₃) m/z (234, MH⁺—NH₃+H⁺), 250 (MH⁺).

### EXAMPLE 2

### 2-Chloro-6-trifluoromethoxy-benzothiazole 2

Into SOCl₂ (6 ml, 82 mmol) preheated to 65° C. was added 1 (0.95 g, 3.81 mmol) slowly (within an hour) and the solution was stirred an additional 1 h at 60° C. SOCl₂ was evaporated, the residue was dissolved in CH₂Cl₂ and solvent evaporated and this treatment of dissolving and evaporating was repeated 4-5 times until almost all SOCl₂ was removed to give crude 2 ready for use in the next step without purification. ¹H-NMR (CDCl₃) δ 7.95 (d, 1H, J=9 Hz, H-4), 7.66 (dq, 1H, J=2, 1 Hz H-7), 7.36 (ddq, 1H, J=9, 2, 1 Hz, H-5); ¹³C (CDCl₃) δ 113.79 (CH, Ar), 120.46 (CF₃O), 120.68, 123.88 (2×CH, Ar), 136.90, 146.91, 149.52 (3×C, C-3a, C-7a, C-6), 154.22 (C-2); MS (CI) (NH₃) m/z (254, M+NH₄⁺).

### EXAMPLE 3

### Methyl-prop-2-ynyl-(6-trifluoromethoxy-benzothiazol-2-yl)-amine 3

Crude 2 (˜2 mmol) was dissolved in N-methyl-propargylamine (˜2 ml, 3.5 eq) and the dark solution was stirred at rt overnight after which a brown solid was obtained and purified by chromatography to give 3. m.p. (for free base)=86-87° C.; ¹H-NMR (CDCl₃) δ 7.56 (d, 1H, J=10 Hz, H-4), 7.49 (dd, 1H, J=2, 1 Hz, H-7), 7.18 (ddq, 1H, J=10, 2, 1 Hz, H-5), 4.4 (d, 2H, J=2.5 Hz, CH₂N), 3.22 (s, 3H, NMe), 2.31 (t, 1H, J=2.5 Hz, PgCH); ¹³C (for free base, CDCl₃) δ 168.61 (C-2), 151.36, 143.38, 131.76 (3×C, C-3a, C-7a, C-6), 120.6 (CF₃O), 119.78, 119.54, 113.89 (3×CH, Ar), 80.21, 73.11 (CH₂CC), 41.49 (NCH₂), 37.82 (NMe); MS (CI)(NH₃) m/z (287, MH+); Anal. (calcd. For C₁₂H₉F₃N₂OS) C, 50.35; H, 3.17; N, 9.79; S, 11.20, found C, 50.62; H, 3.23; N, 9.60; S 11.44.

### EXAMPLE 3a

### Methyl-prop-2-ynyl-(6-trifluoromethoxy-benzothiazol-2-yl)-amine.HCl salt 3a

Free base 3 was dissolved in HCl/EtOH solution, stirred for 0.5 h and the product was precipitated by addition of Et₂O, filtered, washed with Et₂O and dried to give 1.15 g (65%) of 3a. m.p.=160-162.5° C.; ¹H-NMR (DMSO-d₆) δ 7.98 (dd, 1H, J=2 Hz, H-7), 7.58 (d, 1H, J=10 Hz, H-4), 7.32 (ddq, 1H, J=10, 2, 1 Hz, H-5), 4.44 (d, 2H, J=2.5 Hz, CH₂N), 3.39 (t, 1H, J=2.5 Hz, PgCH), 3.18 (s, 3H, NMe); ¹³C (DMSO-d₆) δ 169.09 (C-2), 151.83, 142.66, 132.14 (3×C, C-3a, C-7a, C-6), 120.6 (CF₃O), 119.91, 119.43, 115.06 (3×CH, Ar), 78.69, 75.65 (CH₂CC), 41.42 (NCH₂), 37.95 (NMe); MS (CI)(NH₃) m/z (287, MH⁺).

### EXAMPLE 3b

### Methyl-prop-2-ynyl-(4-trifluoromethoxy-benzothiazol-2-yl)-amine 3b

This compound can be prepared by the same synthetic procedure described for methyl-prop-2-ynyl-(6-trifluoromethoxy-benzothiazol-2-yl)-amine 3, starting from 4-(trifluoromethoxy)-2-benzothiazolamine, reacting it with hydrazinium sulfate and hydrazine hydrate in ethylene glycol, followed by reaction with SOCl₂ and reacting the intermediate with N-methyl-propargylamine.

### EXAMPLE 4

### Prop-2-ynyl-(6-trifluoromethoxy-benzothiazol-2-yl)-amine 4

Propargylamine (0.66 ml, 9.6 mmol, ˜3.5 eq) was added into 2 (obtained from 0.67 g, 2.7 mmol of 1 reacted with 6 ml SOCl₂) and the dark solution obtained was stirred at rt overnight, and then loaded on a chromatography column which resulted in 0.27 g (37%) of 4 as a gray powder. m.p.=138-140° C.; ¹H-NMR (CDCl₃) δ 7.59 (d, 1H, J=10.5 Hz, H-4), 7.49 (dq, 1H, J=2, 1 Hz H-7), 7.21 (ddq, 1H, J=10.5, 2, 1 Hz, H-5), 4.28 (d, 2H, J=2.5 Hz, CH₂N), 2.35 (t, 1H, J=2.5 Hz, CCH); ¹³C (CDCl₃) δ 167.08 (C-2), 148.70, 144.30, 130.32 (3×C, C-3a, C-7a, C-6), 120.49(CF₃O), 120.20, 119.25, 114.29 (3 CH, Ar), 78.19, 73.17 (CH₂CC), 34.69 (NCH₂); MS (CI)(NH₃) m/z (273, MH⁺).

### EXAMPLE 4a

### Prop-2-ynyl-(6-trifluoromethoxy-benzothiazol-2-yl)-amine.HCl salt 4a

0.245 g of 4 were dissolved in isopropanol and Et₂O. EtOH/HCl was added until pH=1. After a few minutes of stirring at rt the mixture was evaporated to dryness and the residue dried in vacuum overnight to give the HCl salt of 4. m.p.=180-181° C.; ¹H-NMR (DMSOd₆) δ 7.95 (d, 1H, J=2 Hz, H-7), 7.60 (d, 1H, J=10 Hz, H-4), 7.33 (dd, 1H, J=10, 2 Hz, H-5), 4.35 (d, 2H, J=2 Hz, CH₂N), 3.37 (t, 1H, J=2 Hz, PgCH); MS (CI)(NH₃) m/z (273, MH+); Anal. (calcd. for C₁₁H₇F₃N₂OS.HCl) C, 42.80; H, 2.61; N, 9.07; S, 10.39; Cl, 11.48; found C, 42.86; H, 2.74; N, 9.03; S, 10.77; Cl, 11.30.

### EXAMPLE 5

### But-2-ynyl-(6-trifluoromethoxy-benzothiazol-2-yl)-amine 5

Compound 5 was obtained by reacting 2 (0.91 g, 3.60 mmol) with but-2-ynylamine (0.93 g, 13.38 mmol, 3.7 eq). Chromatography of the crude product (silica gel, hexane then 10% EtOAc/hexane (200 ml) and finally 20% EtOAc/hexane) gave 0.5 g (65%) of a white solid. ¹H-NMR (DMSO-d₆) δ 8.47 (t, 1H, J=5 Hz, NH), 7.82 (dd, 1H, J=2, 1 Hz, H-7), 7.48 (d, 1H, J=9 Hz H-4), 7.21 (ddq, 1H, J=9, 2, 1 Hz, H-5), 4.15 (dq, 2H, J=5, 2 Hz, CH₂N), 1.80 (t, 3H, J=2 Hz, CCMe); ¹³C (DMSOd₆) δ 166.77 (C-2), 151.38, 142.34, 131.61 (3×C, C-3a, c-7A, c-6), 119.11, 118.59, 114.58 (3×CH, Ar), 78.96, 75.83 (CH₂CC), 33.20 (NCH₂), 3.08 (CCMe).

### EXAMPLE 5a

### But-2-ynyl-(6-trifluoromethoxy-benzothiazol-2-yl)-amine.HCl salt 5a

0.255 g of 5 were converted to the HCl salt using HCl/EtOH as described for 3a. ¹H-NMR (DMSO-d₆) δ 9.75 and 9.07 (two br s, 2×NH), 7.94 (br s, 1H, H-7), 7.59 (br d, 1H, J=9 Hz, H-4), 7.33 (br d, 1H, J=9 Hz, H-5), 4.30 (br s, 2H, CH₂N), 1.83 (br s, 3H, J=2 Hz, CCMe); ¹³C (DMSOd₆) δ 167.19 (C-2), 146.13, 143.18, 129.35 (3×C, C-3a, C-7a, C-6), 120.0 (CF₃O),119.88, 117.36, 115.39 (3×CH, Ar), 80.06, 74.60 (CH₂CC), 34.05 (NCH₂), 3.11 (CCMe); HRMS(FAB+) 287.0258 (MH⁺, 100), 218.1281 (MH⁺—C₄H₅NH₂, 15).

### EXAMPLE 6

### Methyl-(1-methyl-prop-2-ynyl)-(6-trifluoromethoxy-benzothiazol-2-yl)-amine 6

Compound 6 was obtained by reacting 2 (6.82 g, 27 mmol) with N-Me-3-butyn-2-amine (11.0 g, 132.3 mmol, ˜5 eq). Flash chromatography column of the crude product (silica gel, 10% EtOAc/hexane) gave 4.92 g (60.7%) of a thick and almost transparent oil which solidified when placed in freezer. m.p.=42-43° C.; ¹H-NMR (CDCl₃) δ 7.52 (d, 1H, J=9 Hz, H-4), 7.48 (dd, 1H, J=2, 0.5 Hz, H-7), 7.16 (ddq, 1H, J=9, 2, 0.5 Hz, H-5), 5.42 (dq, 1H, J=7, 2 Hz, MeCHNe), 3.13(s, 3H, NMe), 2.38 (d, 1H, J=2 Hz, CCH), 1.51 (d, 3H, J=7 Hz, MeCH); ¹³C (CDCl₃) δ 168.41 (C-2), 151.43, 143.36, 131.33 (3×C, C-3a, C-7a, C-6), 120.6 (CF₃O), 119.76, 119.30, 113.86 (3×CH, Ar), 81.64, 72.70 (CHCC), 47.08 (MeCHNMe), 33.12 (NMe), 19.29 (CHMe); HRMS(FAB+) 301.1118 (MH⁺, 100), 285.0972 (M−Me, 68); Anal. (calcd. for C₁₃H₁₁N₂OSF₃) C, 51.99; H, 3.69; N, 9.33; S, 10.68; found C, 52.07; H, 3.83; N, 9.26; S, 10.67.

### EXAMPLE 7

### Di-prop-2-ynyl-(6-trifluoromethoxy-benzothiazol-2-yl)-amine 7

Into a solution of prop-2-ynyl-(6-trifluoromethoxy-benzothiazol-2-yl)-amine (the compound of Ex. 4, 1.5 g, 5.5 mmol) in acetonitrile (40 ml) were added K₂CO₃ (0.78 g, 5.6 mmol) and propargyl bromide (0.68 g, 0.43 ml, 5.7 mmol), and the heterogeneous mixture was stirred at rt for 4 days. The solid was filtered off and the filtrate evaporated to give a yellow solid which was purified by chromatography (hexane/EtOAC) to give 7 as a white solid (0.62 g, 36%).

¹H NMR (CDCl₃) δ 7.58 (d, 1H, J=9 Hz, H-4), 7.50 (d, 1H, J=1.5 Hz, H-7), 7.18 (dd, 1H, J=9, 1.5 Hz, H-5), 4.45 (d, 4H, J=2 Hz, NCH₂), 2.34 (t, 2H, J=2 Hz, CCH).

¹³C NMR δ: 167.56 (C-2), 150.99, 143.93, 132.02 (C-3a, C-7a, C-6), 120.2 (CF₃O), 120.16, 119.92, 113.99 (3 CH, Ar), 77.12, 73.68 (CH₂CC), 39.33 (NCH₂).

MS (DCI, CH₄) m/z 310 (M), 271 (M−CH₂CC)

### EXAMPLE 8

### (2-Methylsulfanyl-ethyl)-prop-2-ynyl-(6-trifluoromethoxy-benzothiazol-2-yl)-amine 8

The title compound was prepared by the procedure described in Ex. 7: prop-2-ynyl-(6-trifluoromethoxy-benzothiazol-2-yl)-amine (the compound of Ex. 4, 1.35 g, 4.96 mmol) was reacted with 2-chloroethyl methylsulfide (0.49 ml, 4.9 mmol) to give 8 as a white solid (0.61 g, 36%).

¹H NMR (CDCl₃) δ: 7.55 (d, 1H, J=9 Hz, H-4), 7.48 (dq, 1H, J=2, 1 Hz, H-7), 7.17 (ddq, 1H, J=9, 2, 1 Hz, H-5), 4.44 (d, 2H, J=2 Hz, NCH₂CC), 3.83 (t, 2H, J=7 Hz, NCH₂CH₂S), 2.89 (t, 2H, J=7 Hz, NCH₂CH₂S), 2.34 (t, 1H, J=2 Hz, CCH), 2.21 (s, 3H, SMe).

¹³C NMR δ: 167.57 (C-2), 151.09, 143.65, 131.45 (C-3a, C-7a, C-6), 120.2 (CF₃O), 119.89, 119.67, 113.94 (3 CH, Ar), 77.80, 73.46 (CH₂CC), 50.68 (NCH₂CH₂S), 40.38 (NCH₂CC)), 31.39 (NCH₂CH₂S), 15.71 (SMe).

MS (DCI)(CH₄) m/z 346 (M), 300 (M−SMe), 285 (M−CH₂SMe) 272 (M−CH₂ CH₂SMe).

### EXAMPLE 9

### 2-[Prop-2-ynyl-(6-trifluoromethoxy-benzothiazole-2-yl)-amino]-1-p-tolyl-ethanone 9

The title compound was prepared by the procedure described in Ex. 7: prop-2-ynyl-(6-trifluoromethoxy-benzothiazol-2-yl)-amine (the compound of Ex. 4, 5.0 g, 18.3 mmol) was reacted with 2-bromo-4′-methylacetophenone (3.9 g, 18.3 mmol) to give 9 as a white solid (3 g, 40%).

¹H NMR (CDCl₃) δ: 7.91 (d, 2H, J=8 Hz, COAr), 7.50 (d, 1H, J=9 Hz, H-4), 7.45 (d, 1H, J=2 Hz, H-7), 7.29 (d, 2H, J=8 Hz, COAr), 7.14 (ddq, 1H, J=9, 2, 1 Hz, H-5), 5.17 (s, 2H, CH₂CO), 4.47 (d, 2H, J=2 Hz, NCH₂CC), 2.43 (s, 3H, Ar—CH₃), 2.36 (t, 1H, J=2 Hz, CCH).

¹³C NMR δ: 193.20 (CO), 168.21 (C-2), 151.15, 145.01, 143.60, 132.32, 131.81 (5×C, Ar), 129.56, 128.07 (4×CH MeC₆H₄), 120.2 (CF₃O), 119.77, 113.90 (2 CH, Ar), 77.42, 74.20 (CH₂CC), 54.89 (NCH₂CO), 41.15 (NCH₂), 21.74 (MeC₆H₄).

MS (TOF, ES⁺): 405 (MH⁺).

### EXAMPLE 10

### (3-Methyl-6-trifluoromethoxy-3H-benzothiazol-2-ylidene)-prop-2-ynyl-amine 10

2-Imino-3-methyl-6-trifluoromethoxybenzothiazoline (HI salt, 3.26 g, 8.66 mmol) was suspended in toluene (˜70 ml) and propargylamine (13 ml, 0.2 mole) and p-TsOH (0.54 g, 2.88 mmol) were added and the mixture stirred at 125° C. overnight. Toluene was evaporated and the residue purified on chromatography column (silica gel, hexane, 5% EtOAc/hexane and finally 10% EtOAc/hexane) to give 1.75 g of a white solid.

¹H-NMR (CDCl₃) δ 7.26 (dq, 1H, J=2, 1 Hz, H-7), 7.14 (ddq, 1H, J=8, 2, 1 Hz, H-5), 6.86 (d, 1H, J=8 Hz, H-4), 4.02 (d, 2H, J=2 Hz, CH₂N), 3.46 (s, 3H, NMe), 2.28 (t, 1H, J=2 Hz, PgCH); ¹³C 158.64 (C-2), 143.20, 139.65, 118.80 (3×C, C-3a, C-7a, C-6), 122.7 (CF₃O), 119.81, 115.71, 108.88 (3×CH, Ar), 80.69, 70.98 (CH₂CC), 42.20 (NCH₂), 30.49 (NMe); MS (DCI) (CH₄) m/z (285, M−H.), (271, M−CH₃).

### EXAMPLE 11

### (3-Methyl-6-trifluoromethoxy-3H-benzothiazol-2-ylidene)-prop-2-ynyl-amine. HI salt 11

Prop-2-ynyl-(6-trifluoromethoxy-benzothaizol-2-yl)-amine (the compound of example 4, 0.3 g, 1.1 mmole) was dissolved in methyl ethyl ketone, (3 ml) and excess MeI (2 ml) was added. The reaction mixture was refluxed for 4.5 h and then an additional amount of MeI (1-2 ml) was added and the reaction mixture was refluxed over night. The mixture was concentrated, toluene was added and the white precipitate obtained, filtered, washed extensively with toluene and dried in vacuum to give 0.285 g (63%) of 11 as a white solid. ¹H-NMR (CDCl₃) δ 7.66 (dq, 1H, J=2, 1 Hz, H-7), 7.57 (d, 1H, J=9 Hz, H-4), 7.50 (ddq, 1H, J=9, 2, 1 Hz, H-4), 4.50 (d, 2H, J=2 Hz, CH₂N), 4.25 (s, 3H, NMe), 2.49 (t, 1H, J=2 Hz, PgCH); ¹³C (DMSOd₆) 168.24 (C-2), 144.57, 138.61, 118.31 (3×C, C-3a, C-7a, C-6), 122.58 (CF₃O), 121.59, 116.98, 114.74 (3×CH, Ar), 77.32, 76.46 (CH₂CC), 36.26 (NCH₂), 33.11 (NMe); TOF MS ES m/z (287, MH⁺).

### EXAMPLE 12

### (3-Methyl-4-trifluoromethoxy-3H-benzothiazol-2-ylidene)-prop-2-ynyl-amine 12

The title compound may be prepared from 2-imino-3-methyl-4-trifluoromethoxybenzothiazoline (prepared from 4-(trifluoromethoxy)-2-benzothiazolamine) by reacting it in toluene with propargylamine in the presence of p-TsOH at 125° C. overnight.

### EXAMPLE 13

### 2-(2-Prop-2-ynylimino-6-trifluoromethoxy-benzothiazole-3-yl)-1-p-tolyl-ethanone 13

Prop-2-ynyl-(6-trifluoromethoxy-benzothiazol-2-yl)-amine (the compound of Ex. 4, 0.58 g, 2.14 mmol) was dissolved in acetonitrile (40 ml) and 2-bromo-4′-methylacetophenone (0.51 g, 2.14 mmol) was added, and the reaction mixture was refluxed for 48 h. The solvent was evaporated and the crude residue obtained was purified by chromatography to give 13 as a solid,

mp: 98-100° C. (0.6 g, 6%).

¹H NMR (DMSOd₆) δ: 7.97 (d, 2H, J=8 Hz, COAr), 7.80 (d, 1H, J=1 Hz, H-7), 7.40 (d, 1H, J=8 Hz, COAr), 7.23 (dd, 1H, J=8, 1 Hz, H-5), 7.17 (d, 1H, J=8 Hz, H-4), 5.59 (s, 2H, CH₂CO), 3.93 (d, 2H, J=1.5 Hz, NCH₂CC), 3.12 (t, 1H, J=1.5 Hz, CCH), 2.41 (s, 3H, Ar—CH₃).

¹³C NMR δ: 192.30 (CO), 158.21 (C-2), 145.60, 143.95, 140.83, 133.50, (4×C, Ar), 130.30, 129.09, (4×CH MeC₆H₄), 124.23 (Ar), 120.4 (CH, Ar), 120.0 (CF₃O), 116.83, 110.84 (2 CH, Ar), 81.46, 72.37 (CH₂CC), 50.47 (NCH₂CO), 42.35 (NCH₂), 21.64 (MeC₆H₄).

MS: 404 (M⁺, 43), 352 (7), 285 (75), 119 (100).

### EXAMPLE 14

### Prop-2-ynyl-(3-prop-2-ynyl-6-trifluoromethoxy-3H-benzothiazol-2-ylidene)-amine 14

2-Imino-3-(2-propynyl)-6-trifluoromethoxy-benzothiazoline HBr (2.5 g, 7.08 mmol) was suspended in toluene (40 ml), and propargylamine (10 ml, 0.156 mol) and p-TsOH (0.444 g, 2.33 mmol) were added and the mixture was stirred at 150° C. overnight. The solvent was evaporated and the dark residue was purified by chromatography to give 14 as a white solid (0.15 g, 7%).

¹H NMR (CDCl₃) δ: 7.28 (d, 1H, J=1.5 Hz, H-7), 7.18 (ddq, 1H, J=9, 1.5, 1 Hz, H-5), 7.07 (d, 1H, J=9 Hz, H-4), 4.80 (d, 2H, J=2 Hz, NCH₂), 4.06 (d, 2H, J=2 Hz, SCNCH₂), 2.29 (t, 1H, J=2.5 Hz, CCH), 2.27 (t, 2H, J=2 Hz, CCH).

¹³C NMR δ: 157.14 (C-2), 143.64, 138.00, 123.52 (C-6, C-3a, C-7a), 120.2 (CF₃O), 119.84, 115.84, 109.75 (3 CH, Ar), 80.44, 76.80 (CH₂CC), 42.12 (SCNCH₂), 32.76 (NCH₂).

MS (DCI, CH₄): m/z 311(M⁺).

### EXAMPLE 15

### [3-(2-Methylsulfanyl-ethyl)-6-trifluoromethoxy-3H-benzothiazol-2-ylidene]-prop-2-ynyl-amine 15

3-(2-Methylthio ethyl)-2-imino-6-trifluoromethoxy-benzothiazoline HCl (1 g, 2.9 mmol) was suspended in toluene (25 ml), and propargylamine (4.5 ml, 4 g, 70 mmol) and p-TsOH (0.185 g, 1 mmol) were added and the mixture was stirred at 130-135° C. under a nitrogen atmosphere for 24 h. The solvent was evaporated and the dark residue was purified by chromatography to give 15 as a viscous oil (0.1 g, 10%).

¹H NMR (CDCl₃) δ: 7.28 (dd, 1H, J=1, 1.5 Hz, H-7), 7.11 (ddq, 1H, J=9, 1.5, 1 Hz, H-5), 7.07 (d, 1H, J=9 Hz, H-4), 4.18 (t, 2H, J=7 Hz, NCH₂), 4.04 (d, 2H, J=2 Hz, SCNCH₂), 2.82 (t, 2H, J=7 Hz, CH₂SMe), 2.26 (t, 1H, J=2 Hz, CCH), 2.19 (s, 3H, SMe).

¹³C NMR δ: 157.64 (C-2), 143.21, 138.79, 123.53 (C-6, C-3a, C-7a), 120.2 (CF₃O), 119.75, 115.86, 109.04 (3 CH, Ar), 80.50, 70.92 (CH₂CC), 43.39 (SCNCH₂), 42.13 (NCH₂), 30.60 (CH₂SMe),15.78 (SMe).

MS (DCI, i-Bu) m/z: 347 (M⁺), 272 (M⁺−C₂H₄SMe).

### EXAMPLE 16

### 3-Methyl-2-(methyl-prop-2-ynyl-amino)-6-trifluoromethoxy-benzothiazol-3-ium iodide 16

(3-Methyl-6-trifluoromethoxy-3H-benzothiazol-2-ylidene)-prop-2-ynyl-amine (the compound of example 10, 120 mg, 0.42 mmol) was refluxed in MeI (3-4 ml) for 4 h. Then MEK (1 ml) was added and also additional amount of MeI (1-2 ml) and the reaction was heated up to 75° C. for additional 2 h and then the mixture was stirred at rt for 24 h. Excess MeI and solvent were evaporated to leave a brown-yellow residue which was treated with hexane, filtered and washed with hexane and ether to afford 30 mg (17%) of 16 as a brown solid. ¹H-NMR (DMSOd₆) δ 8.32 (s, 1H, H-7), 7.97 (d, 1H, J=9 Hz, H-4), 7.72 (bd, 1H, J=9 Hz, H-5), 4.67 (d, 2 H, J=1.5 Hz, CH₂N), 4.01 (s, 3H, N⁺Me), 3.83 (bs, 1H, PgCH), 3.52 (s, 3H, NMe); ¹³C (DMSOd₆) 173.27 (C-2), 145.83, 140.06, 124.72 (3×C, C-3a, C-7a, C-6), 122.42, 117.04, 116.73 (3×CH, Ar), 79.53, 76.42 (CH₂CC), 46.61 (NCH₂), 42.86 (N⁺Me), 38.26 (NMe); HRMS (FAB+) 286.0387 (M−MeI, 100).

### EXAMPLE 17

### Effect of 3a on MPP+ Treated PC-12 Cells

Pheochromocytoma PC-12 cells (at a density of 200,000 cells/well) were cultured for 10 days with 50 ng/ml NGF on 6-well culture dishes coated with 200 ug/ml rat tail type I collagen (BD Biosciences, Bedford, Mass., USA). On the day of the experiment the morphological differentiation of the cells was very advanced (typical network formation). In order to initiate the neurotoxic insult, cells were treated with 1000 μM of 1-methyl-4-phenylpyrdinium (MPP+) iodide salt from RBI chemicals (Natick, Mass., USA) for 48 hours in the absence or presence of tested compounds, added to the culture 30 min. prior to MPP+ administration. MPP+ has been shown to inhibit mitochondrial electron transport (complex I) in neurons and to induce a syndrome resembling Parkinson disease in mice and monkeys. At the cellular level, neuronal cell death is induced by several mechanisms including pathological concentrations of intracellular calcium and free oxygen radicals. Therefore the positive control in this experiment was nimodipine at the concentration of 10 μM (RBI chemicals, Natick, Mass., USA)(a potent L-type calcium channel blocker). At the end of the experiment cell death was measured by assessing Lactate dehydrogenase (LDH) activity in the medium. High medium LDH indicated increased neuronal death that promoted leakage of this cytoplasmic enzyme into the medium.

Measuring lactate dehydrogenase activity in the medium was performed using a Sigma Diagnostics LD-L reagent. LDH activity was spectrophotometrically monitored at 340 nm by following the rate of conversion of oxidized nicotinamide adenine dinucleotide (NAD⁺) to the reduced form of (NADH). Total LDH of each culture (extracellular+intracellular) was obtained by measuring LDH in the medium after freezing and thawing of the cultures. Basal LDH release was measured in untreated cultures (no MPP+). The neurotoxic effect was calculated according to the formula: (LDHs−LDHb)/LDHt×100. (s=sample; b=basal; t=total). Each compound was tested in sixplicate.

Results are summarized in the table below. Without MPP+ (control) cell death was very low, not exceeding 1.1% of total. After the MPP+ insult, toxicity reached 49.7% (LDH release as % of total). Pretreatment of the cultures with nimodipine (30 minutes before MPP+), reduced LDH release dramatically to a value of 3.6%, indicative of a strong neuroprotective effect. Pretreatment of cultures with 10 μM 3a, reduced LDH release to a value of 10.9%, suggestive of a strong neuroprotective effect.

TABLE 1
Percent of LDH Release

| | | Percent of total LDH release |
|---|---|---|
| MPP+, 1000 μM | Tested compound | (mean ± S.E.M.) |
| − | — | 1.1 ± 0.2 |
| + | — | 49.7 ± 5 |
| + | Nimodipine, 10 μM | 3.6 ± 0.4 |
| + | 3a, 10 μM | 10.9 ± 2.3 |
| + | 4a, 10 μM | 25 ± 8.0 |

### EXAMPLE 18

### Effect of 3a on MPP+ Toxicity in Mice

MPP+ is a neuro-toxin and its injection into the brain causes depletion of striatal dopamine. Male C₅₇bl mice, weighing 24-25 g were divided into 4 groups A-D receiving the following treatments:

A: Oral administration of 0.25% Carboxymethyl cellulose (CMC, 0.1 ml/mouse) and intracerebroventricular (ICV) injection of saline (10 μl/mouse).

B. Oral administration of 5 mg/kg 3a in 0.1 ml CMC, and ICV injection of 10 μl saline.

C. Oral administration of 0.1 ml CMC, and ICV injection of 30 μg MPP+ dissolved in 10 μl saline.

D. Oral administration of 5 mg/kg 3a in 0.1 ml CMC, and ICV injection of 30 μg MPP+ dissolved in 10 μl saline.

3a/CMC or CMC alone were orally administered 30 minutes before and 2 hours after ICV injection. The injection was done according to Haley T. J and McCormick W. G (Brit.J. Pharmacol., 1957, 12, 12) using ether anesthesia. Saline or MPP+/saline were injected during 1 second using a syringe that delivered the liquid at a rate of 0.6 ml/min. Two ICV injections were carried out with a 24 hour interval between them. Mice were killed six days after the last injection and striata taken for dopamine and DOPAC determinations. Each striatum was weighed, homogenized in 0.1M Perchloric acid (0.6 ml) containing 2 mM sodium metabisulfite and 0.3 mM EDTA. After centrifugation at 13000 g for 7 minutes, the supernatant was taken for catecholamine determination. Aliquotes of 20 μl were injected into the solvent stream of an HPLC apparatus equipped with a Microsorb column (packing 3 μm, 4.6 mm diameter, 12.5 cm long). The mobile phase was composed of NaH₂PO₄ 100 mM, octan-1-sulphonic acid 1.5 mM, disodium ethylenediaminetetracetic acid 250 μM, methanol 2.3%, acetonitrile 4% in grade deionized water, at a flow rate of 1.0 ml min⁻¹. Dopamine and DOPAC were detected with an ESA Coulochem model 5014 detector (Bedford, Mass., USA). Column eluates were initially oxidized by an ESA guard cell (model 5020) at +300 mV, then reduced at +60 mV at detector 1 and measured at detector 2 at −250 mV.

Results are presented in the table below as concentrations of dopamine or DOPAC in pmol per mg striatal tissue. MPP+ caused a depletion of about 30% in striatal concentrations of dopamine and DOPAC (Group C) and 3a reduced this depletion (Group D).

TABLE 2

| | | | Dopamine | DOPAC |
|---|---|---|---|---|
| | | | Pmol/mg | Pmol/mg |
| Group | MPP+ | Drug | tissue +/− S.E.M. | tissue +/− S.E.M. |
| A (N = 9) | − | CMC | 64.7 +/− 8.7 | 4.0 +/− 0.3 |
| B (N = 9) | − | CMC + 3a | 78.2 +/− 4.3 | 4.8 +/− 0.4 |
| C (N = 10) | + | CMC | 45.1 +/− 7.8 | 2.8 +/− 0.4 |
| D (N = 10) | + | CMC + 3a | 66.0 +/− 3.4 | 3.7 +/− 0.2 |

### EXAMPLE 19

### Activity of Compounds of the Invention in the Experimental Allergic Encephalomyelitis (“EAE”) Model of MS

EAE is an accepted animal model of autoimmune disorder (Tisch et al. Proc. Natl. Acad. Sci. USA (1994) 91:437-438). EAE was induced by injecting the encephalitogenic agent consisting of MSCH and commercial CFA containing Mycobacterium tuberculosis H37Ra to the foot-pads of the animals and pertussis toxin intravenously.

Administration of the Test Article:

GA-DS in MSCH emulsion was injected in the four footpads. The other compounds were administered to the respective groups by oral gavage twice daily for 30 consecutive days starting from the day of induction until the termination of the study.

Clinical Signs

Scoring of EAE clinical signs was initiated from Day 10 post-EAE induction and was continued daily for 20 days. The clinical signs were recorded on observation cards according to a grading system described in the table below.

TABLE 3
Evaluation of the EAE clinical signs.

| Score | Signs | Description |
|---|---|---|
| 0 | Normal behavior | No neurological signs. |
| 1 | Tail weakness | The mouse tail is limp and |
| | | droops. |
| 2 | Hind legs | Limb paresis, wobbly walk — |
| | weakness | when the mouse walks the hind |
| | | legs are unsteady. |
| 3 | Hind legs | The mouse can't move its hind |
| | paralysis | legs and it drags them when it |
| | | walks. |
| 4 | Full paralysis | The mouse can't move its legs |
| | | at all, it looks thinner and |
| | | emaciated. |
| 5 | Death | |

Calculation of the Mortality Rate

The number of dead or moribund animals in each group were summed.

The mortality rate was calculated as:

$$\frac{Number of dead or moribund mice in treated group}{Number of dead or moribund mice in control group}$$

Calculation of the Mean Maximal Score and Percent Inhibition

The mean maximal score (MMS) of each group was calculated as Σ maximal score of each mouse/number of mice in the group.

The percent inhibition was calculated as

$$Percent Inhibition = 1 - \frac{( MMS of treated group )}{MMS of control group} \times 100$$

Calculation of the Mean Group Score and Percent Inhibition

The daily scores of each mouse in the test group was summed and the individual mean daily score (IMS) was calculated as IMS=daily score of mouse/observation period (days).

The mean group score (GMS) will be calculated as Σ IMS of each mouse/number of mice in the group.

The percent inhibition will be calculated as

$$Percent inhibition = 1 - \frac{[ GMS of treated group ]}{GMS of control group} \times 100$$

### EXAMPLE 19a

### Combination of Compounds of the Invention with GA

The GA was weighed and diluted in sterilized phosphate buffered saline (PBS) and test compounds in 0.5% methyl-cellulose.

The results for treatment with compound 3 are summarized in FIGS. 1-A, 1-B and 1-C.

### EXAMPLE 19b

### Compounds of the Invention (no GA)

The results are summarized in the Table 4 and in FIGS. 2(a) and 2(b).

Other compounds which were made are tested and show activity comparable with the activity of the compounds which were tested.

TABLE 4
Inhibition of EAE clinical signs when dosed
with 10 mg/kg twice daily.

| | MMS % | GMS % | Mortality % |
|---|---|---|---|
| Compound # | Inhibition | Inhibition | Inhibition |
| 3 | 73.5 | 87.5 | 100 |
| 9 | 48.8 | 57.6 | 50 |
| 10 | 34.1 | 37.5 | 40 |

What is claimed is:

1. A compound having the structure: 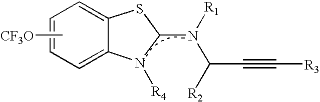
wherein
R₁ is present or absent, and when present is H, C₁-C₆ alkyl, C₁-C₆ alkynyl, —(CH₂)_{y}S(CH₂)ₓCH₃, C₁-C₆ aminoalkyl, C₁-C₆ hydroxyalkyl or —(CH₂)ₙC(═O)(C₆H₄)(CH₂)R₂;
R₂ is H or C₁-C₄ alkyl;
R₃ is H or C₁-C₄ alkyl;
R₄ is present or absent, and when present is H, C₁-C₆ alkyl, C₁-C₆ alkynyl, —(CH₂)_{y}S(CH₂)ₓCH₃, C₁-C₆ aminoalkyl, C₁-C₆ hydroxyalkyl or —(CH₂)ₙC(═O)(C₆H₄)(CH₂)R₂;
wherein n is an integer from 1-6;
wherein x is 0 or an integer from 1-5 and y is an integer from 1-5, such that x+y<6;
at least one of R₁ or R₄ is present;
the dashed line represents a bond between one of the nitrogen atoms and the intervening carbon atom; and
the compound is charged when both R₁ and R₄ are present,
or a specific enantiomer thereof or a pharmaceutically acceptable salt thereof.

2. The compound of claim 1, wherein at least one of R₁ or R₄ is —(CH₂)ₙC(═O)(C₆H₄)(CH₂)R₂.

3. The compound of claim 1, wherein at least one of R₁ and R₄ is —(CH₂)_{y}S(CH₂)ₓCH₃.

4. The compound of claim 1, having the structure: 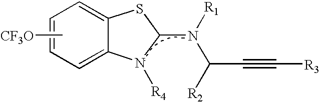
wherein
R₁ is present or absent, and when present is H or C₁-C₄ alkyl;
R₂ is H or C₁-C₄ alkyl;
R₃ is H or C₁-C₄ alkyl;
R₄ is present or absent, and when present is H or C₁-C₄ alkyl;
at least one of R₁ or R₄ is present;
the dashed line represents a bond between one of the nitrogen atoms and the intervening carbon atom; and
the compound is charged when both R₁ and R₄ are present,
or a specific enantiomer thereof or a pharmaceutically acceptable salt thereof.

5. The compound of claim 4, having the structure:

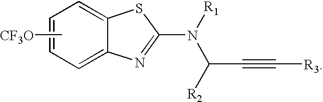

6. The compound of claim 4, having the structure:

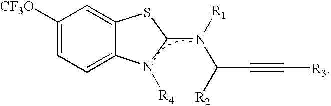

7. The compound of claim 4, having the structure:

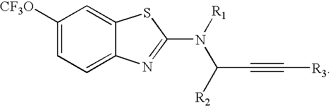

8. The compound of claim 4, 5, 6 or 7 wherein at least one of R₁, R₂ and R₃ is C₁-C₄ alkyl.

9. The compound of claim 4 or 6, wherein R₁ is absent and R₄ is present.

10. The compound of claim 4, 5, 6 or 7 wherein the chiral carbon is in the R configuration.

11. The compound of claim 4, 5, 6 or 7 wherein the chiral carbon is in the S configuration.

12. The compound of claim 9, wherein R₁ is absent and R₄ is methyl.

13. The compound of claim 7, wherein
R₁ is H or methyl;
R₂ is H or methyl;
R₃ is H or methyl,
or a pharmaceutically acceptable salt thereof.

14. The pharmaceutically acceptable salt of the compound of any one of claims 1-13, wherein the salt is the chloride, mesylate, maleate, fumarate, tartarate, hydrochloride, hydrobromide, esylate, p-toluenesulfonate, benzoate, acetate, phosphate or sulfate salt.

15. The compound of claim 2 having the structure:

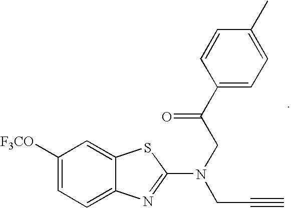

16. The compound of claim 1 having the structure:

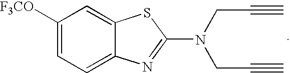

17. The compound of claim 3 having the structure:

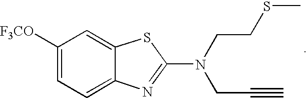

18. The compound of claim 3 having the structure:

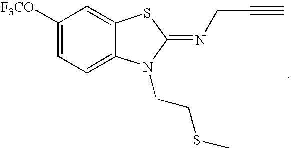

19. The compound of claim 1 having the structure:

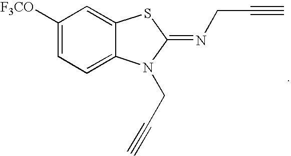

20. The compound of claim 2 having the structure:

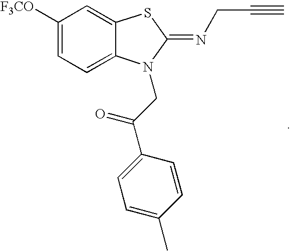

21. The compound of claim 7, having the structure:

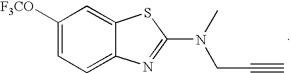

22. The hydrochloride salt of the compound of claim 21.

23. The compound of claim 7, having the structure:

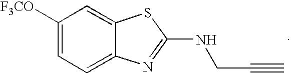

24. The hydrochloride salt of the compound of claim 23.

25. The compound of claim 7, having the structure:

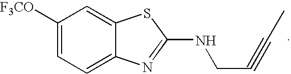

26. The hydrochloride salt of the compound of claim 25.

27. The compound of claim 7, having the structure:

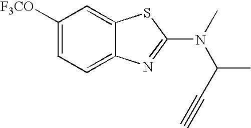

28. The hydrochloride salt of the compound of claim 27.

29. The compound of claim 5, having the structure:

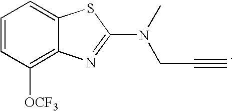

30. The hydrochloride salt of the compound of claim 29.

31. The compound of claim 6, having the structure:

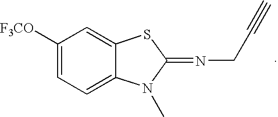

32. The hydrochloride salt of the compound of claim 31.

33. The compound of claim 4, having the structure:

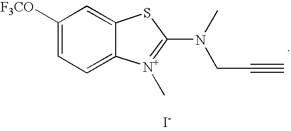

34. The compound of claim 4, having the structure:

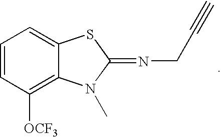

35. A method for treating a subject afflicted with a neurologic disorder comprising administering to the subject a therapeutically effective amount of a compound of claim 1 or a pharmaceutically acceptable salt thereof, so as to thereby treat the neurologic disorder in the subject.

36. The method of claim 35, wherein the neurologic disorder is Parkinson's Disease, Alzheimer's Disease, amyotrophic lateral sclerosis, stroke, a neuromuscular disorder, schizophrenia, cerebral infarction, head trauma, glaucoma, facialis or Huntington's Disease.

37. The method of claim 35, wherein the therapeutically effective amount is from about 1 to about 1000 mg/day.

38. A method for treating a subject afflicted with multiple sclerosis comprising administering to the subject a therapeutically effective amount of a compound of claim 1 or a pharmaceutically acceptable salt thereof so as to thereby treat multiple sclerosis in the subject.

39. The method of claim 38, further comprising administering to the subject a therapeutically effective amount of levodopa, glatiramer acetate, interferon beta-1b, interferon beta-1a, steroids or Mitoxantrone.

40. The method of claim 38, wherein the therapeutically effective amount is from about 1 to about 1000 mg/day.

41. The method of claim 35 or 38 wherein the therapeutically effective amount of the compound is administered by injection, systemically, orally or nasally.

42. A method for destroying or inhibiting the proliferation of microbes or fungus which comprises contacting the microbes or fungus with a composition comprising the compound of claim 1 and an acceptable carrier.

43. A pharmaceutical composition comprising the compound of claim 1 and a pharmaceutically acceptable carrier.

44. The pharmaceutical composition of claim 43, further comprising a therapeutically effective amount of levodopa, glatiramer acetate, interferon beta-1b, interferon beta-1a, steroids or Mitoxantrone.

45. The pharmaceutical composition of claim 43, further comprising a therapeutically effective amount of glatiramer acetate.

46. A process for the manufacture of a pharmaceutical composition comprising admixing the compound of claim 1 with a pharmaceutically acceptable carrier.

47. A packaged pharmaceutical composition for treating a neurologic disorder in a subject comprising:
(a) the pharmaceutical composition of claim 43; and
(b) instructions for using the composition for treating the neurologic disorder in the subject.

48. A process of manufacturing the compound of claim 4 comprising the steps of:
(a) reacting 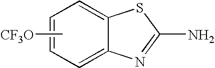
under suitable conditions with an amine exchanging agent in the presence of solvent to provide: 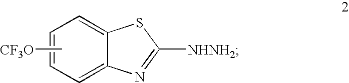
(b) treating 2 with a chlorinating agent to provide 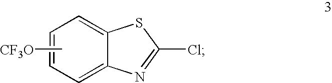
(c) reacting 3 with 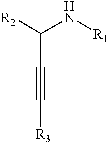
to provide 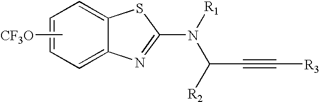
wherein
R₁ is present or absent, and when present is H or C₁-C₄ alkyl;
R₂ is H or C₁-C₄ alkyl;
R₃ is H or C₁-C₄ alkyl; and
(d) optionally alkylating the product of step (c), wherein R₁ is H, to provide the compound.

49. The process of claim 48, further comprising reacting the product of step (c), wherein R₁, R₂ and R₃ are each H, with 2-bromo-4′-methylacetophenone in a polar solvent in the presence of a base to produce a compound having the structure:

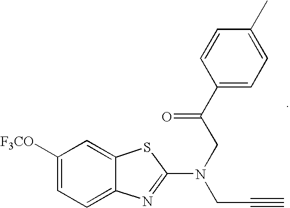

50. The process of claim 49, wherein the polar solvent is acetonitrile and the base is potassium carbonate.

51. The process of claim 48, further comprising reacting the product of step (c), wherein R₁, R₂ and R₃ are each H, with propargyl bromide in a polar solvent in the presence of a base to produce a compound having the structure:

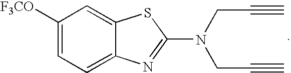

52. The process of claim 51, wherein the polar solvent is acetonitrile and the base is potassium carbonate.

53. The process of claim 48, further comprising reacting the product of step (c), wherein R₁, R₂ and R₃ are each H, with 2-chloroethyl methylsulfide in a polar solvent in the presence of a base, to produce a compound having the structure:

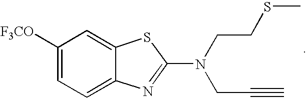

54. The process of claim 53, wherein the polar solvent is acetonitrile and the base is potassium carbonate.

55. The process of claim 48, wherein the amine exchanging agent is a mixture of aqueous NH₂NH₂ and hydrazinium sulfate in ethylene glycol.

56. The process of claim 55, wherein the chlorinating agent is SOCl₂.

57. The process of claim 56, wherein R₁ is C₁-C₄ alkyl and R₂ and R₃ are H.

58. The process of claim 48, wherein the alkylating agent in step (d) is methyliodide or dimethyl sulfate.

59. A process of manufacturing a compound having the structure: 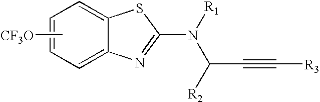
wherein
R₁ is C₁-C₄ alkyl;
R₂ is H or C₁-C₄ alkyl; and
R₃ is H or C₁-C₄ alkyl,
comprising reacting a compound having the structure: 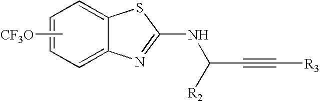
with R₁X in a polar solvent in the presence of a base, wherein X is a halogen atom, to produce the compound.

60. The process of claim 59, wherein the polar solvent is acetonitrile and the base is potassium carbonate.

61. A process of manufacturing a compound having the structure: 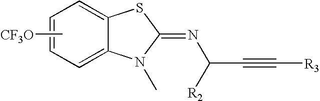
wherein
R₂ is H or C₁-C₄ alkyl; and
R₃ is H or C₁-C₄ alkyl,
comprising,
a) reacting 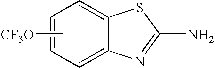
under suitable conditions with a methylating agent, in the presence or absence of solvent to provide: 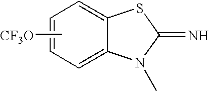
b) reacting the product of step a) with 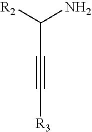
in the presence of p-toluenesulfonic acid to provide the compound.

62. The process of claim 61, wherein the product of step (b) is further alkylated with an alkylating agent to provide a compound having the structure:

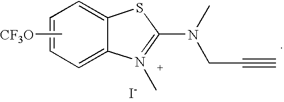

63. The process of claim 61, wherein the methylating agent in step (a) is methyliodide or dimethyl sulfate.

64. The process of claim 62 wherein the methylating agent is methyliodide.

65. A process of manufacturing the compound of claim 19 comprising reacting a compound having the structure: 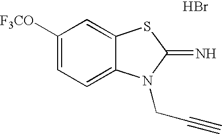
with propargylamine and p-TsOH in toluene to produce the compound.

66. A process of manufacturing the compound of claim 18 comprising reacting a compound having the structure: 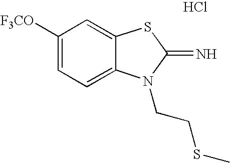
with propargylamine and p-TsOH in toluene to produce the compound.

67. A process of manufacturing the compound of claim 20 comprising reacting a compound having the structure: 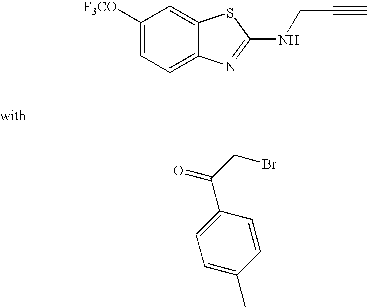
in a polar solvent to produce the compound.

68. The process of claim 67, wherein the polar solvent is acetonitrile.

69. Use of the compound of any one of claims 1-34 for manufacturing a medicament useful for treating a neurologic disorder in a subject.

70. The use of claim 69, wherein the neurologic disorder is Parkinson's Disease, Alzheimer's Disease, amyotrophic lateral sclerosis, stroke, a neuromuscular disorder, schizophrenia, cerebral infarction, head trauma, glaucoma, facialis or Huntington's Disease.

71. Use of the compound of any one of claims 1-34 for manufacturing a medicament useful for treating multiple sclerosis in a subject.

72. The use of claim 71, wherein the medicament further comprises levodopa, glatiramer acetate, interferon beta-1b, interferon beta-1a, steroids or Mitoxantrone.

73. Use of the compound of any one of claims 1-34 for manufacturing a medicament in a package having instructions for administration of the medicament to treat a neurologic disorder in a subject.
